(19) United States
(12) Patent Application Publication
(10) Pub. No.: US 2021/0161734 A1
(43) Pub. Date: Jun. 3, 2021

(54) Absorbent Article With Low Profile Closure

(71) Applicant: 3M INNOVATIVE PROPERTIES COMPANY, St. Paul, MN (US)

(72) Inventors: Thomas J. Gilbert, St. Paul, MN (US); Michael R. Gorman, Woodbury, MN (US); Peter Kitzer, Echt (NL)

(21) Appl. No.: 17/250,474

(22) PCT Filed: Jul. 31, 2019
(86) PCT No.: PCT/IB2019/056542

§ 371 Date: Jan. 26, 2021

Related U.S. Application Data
(60) Provisional application No. 62/713,170, filed on Aug. 1, 2018.

(51) Int. Cl. A61F 13/56; A61F 13/49

(52) U.S. Cl. CPC A61F 13/5644; A61F 2013/15869; A61F 13/49015; A61F 13/5655

(57) ABSTRACT

An absorbent article includes an absorbent chassis and a fastening system that together define a refastenable pant. The refastenable pant includes a pair side panels comprising a front side panel and aback side panel. The front side panel and back side panel forming a leg opening when fastened together. The refastenable pant comprising a fastening system that includes bridging components that refastenably connect the front side and back side panels in the manner of a butt-joint thus producing a side panel with a low profile seam appearance.

### BACKGROUND

Training pants are particularly useful in the toilet training process because children can associate the transition from diapers to training pants with “growing up” or progression toward the goal of wearing underwear. Furthermore, pant style diapers allow the child to be able to pull the diaper down before using the toilet and to pull them back up when finished, therefore allowing the child the independence and confidence to successfully toilet train.

Many traditional training pants are provided with permanent side seams requiring the pant to be pulled on and off like a regular undergarment. Some training pants are provided with refastenable side seams, allowing the pant to be applied like a pant or like a diaper, the latter conferring the ability to apply the pant in a manner of diaper or a pull-up.

### SUMMARY OF THE INVENTION

The present invention relates, in part, to an absorbent article, comprising an absorbent chassis having a front portion, back portion, and crotch portion the crotch portion extending between and connecting the front portion and back portion, the absorbent chassis having first and second major longitudinal edges, first and second laterally opposed side panels, connecting the front portion to the back portion along a length of the first and second major longitudinal edges, to define first and second leg openings in the absorbent article, wherein the first and second side panels comprise a front side panel having a distal edge, a rear side panel having a distal edge, and a bridging component releasably coupling the front and rear side panels in a non-overlapping configuration.

Other aspects of the invention, relate, in part to a method of making an absorbent article, the method comprising: coupling first and second side panels to an absorbent chassis, the absorbent chassis having a front portion, back portion, and crotch portion, the crotch portion extending between and connecting the front portion and back portion, and having first and second major longitudinal edges, and wherein the first and second side panels are coupled to absorbent chassis along lengths of the first and second major longitudinal edges, to define first and second leg openings in the absorbent article; wherein the first and second side panels comprise a front side panel having a distal edge, a rear side panel having a distal edge, and a bridging component coupling the front and rear side panels in a non-overlapping configuration.

In yet other aspects, the present invention relates, in part, to a side panel for a pull-up type absorbent article, comprising a first panel having first and second major surfaces and distal edge, a second panel having first and second major surfaces and a distal edge, a bridging component coupled to the first major surface of the first panel and the first major surface of the second panel—wherein the bridging component holds the distal edges of the first and second panels together in a butt-joint configuration.

### BRIEF DESCRIPTION OF DRAWINGS

FIG. 1 is a side view of a typical prior art disposable absorbent article with side seams.

FIG. 2 is a plan view of the absorbent article shown in FIG. 1 in an unfastened, open, laid out condition.

FIG. 3 is a plan view of an embodiment of the bridging component of the invention.

FIG. 4A is a schematic of one embodiment of an improved side seam pant closure.

FIG. 4B is a schematic of one embodiment of an improved side seam pant closure.

FIG. 4C is a schematic of one embodiment of an improved side seam pant closure.

FIG. 4D is a schematic of one embodiment of an improved side seam pant closure.

FIG. 4E is a schematic of one embodiment of an improved side seam pant closure.

FIG. 5 is a schematic of an embodiment of the bridging component.

FIG. 6A is a schematic of a prior art side seam.

FIG. 6B is a schematic of a side seam achieved employing an embodiment an improved bridging component.

FIG. 7 is perspective view of an embodiment of an improved bridging component shown in a folded orientation.

FIG. 8 is side view of a pant type diaper employing an embodiment of the bridging component of the invention.

FIG. 9 is a plan view of the absorbent article shown in FIG. 8 in an unfastened, open, laid out condition.

FIG. 10 is a side view schematic of an embodiment of a side seam according to an embodiment of the invention.

### DETAILED DESCRIPTION

Traditional training pants are used by toddlers when transitioning between diapers and knit underwear. They have absorbent capabilities along the lines of diapers, but whereas a diaper is traditionally constructed to wrap around the underside of an infant and then be secured with the use of lateral side closure fasteners of some sort (for example adhesive tabs or the like), training pants are structured more like underwear and do not include side closure fasteners. However, to facilitate easy removal of traditional training pants, a removal seam is typically disposed on either side of the training pant, allowing a user to rip the training pant away from the wearer, thus splitting the sides of the training pant along the removal seam.

A problem associated with some training pants of the prior art is that the removal seams are an overlap design, giving traditional training pants a more diaper-like appearance resembling closure tabs, thereby potentially frustrating toilet training efforts due to the child's perception that he or she is wearing a diaper. It would be desirable to have a disposable absorbent article that provides the garment-like or underwear-like look of a traditional training pant yet affords the option of being applied either like a diaper or like a pant.

The principles of the present invention can be incorporated into any suitable disposable absorbent article. Examples of such suitable articles include diapers, training pants, feminine hygiene products, incontinence products, other personal care or health care garments, or the like. For ease of explanation, the description hereafter will be in terms of a child's training pant.

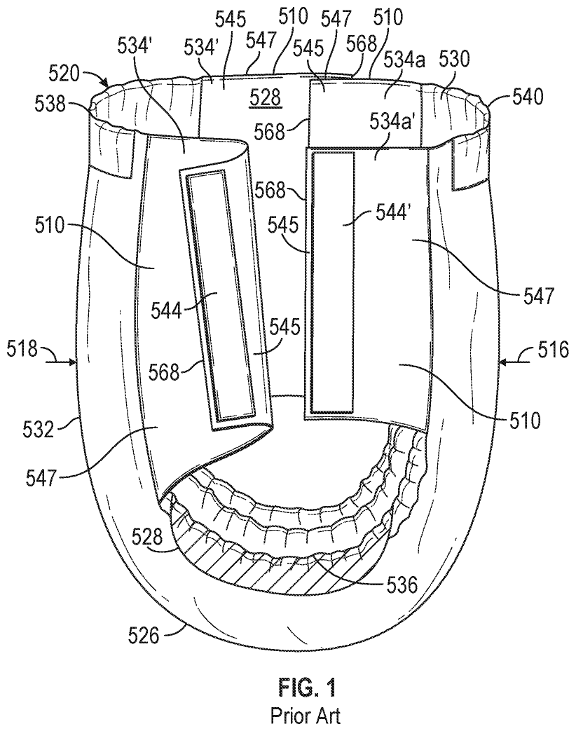

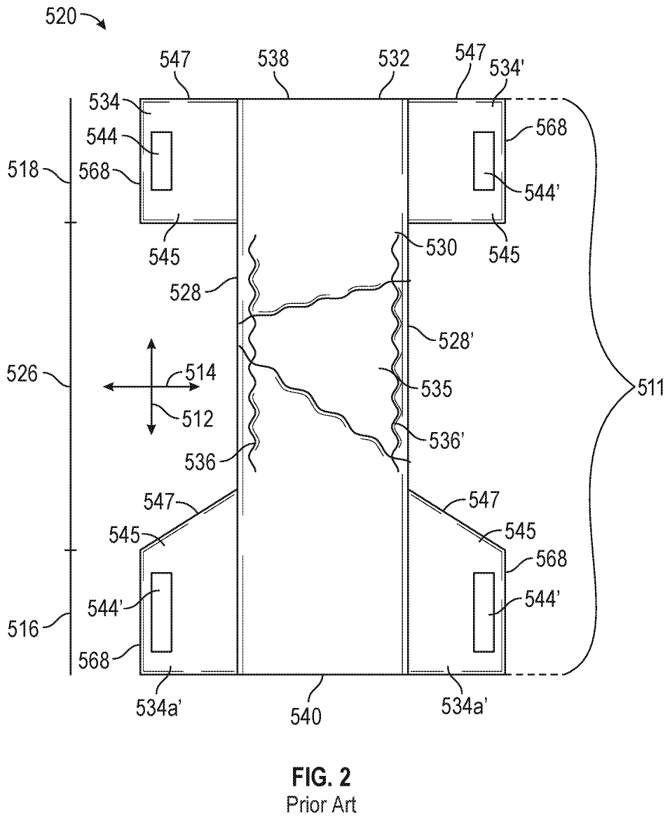

FIG. 1 and FIG. 2 illustrate a training pant 520 of the prior art, FIG. 1 shown in partially fastened, three dimensional view, FIG. 2 shown in plan view (unfastened). Reference elements are interchangeable between the two figures. Referring to FIG. 2, training pant 520 has a longitudinal direction 12 and a lateral direction 14. In the longitudinal direction 12, the training pant 520 defines an absorbent chassis 511 that can be divided into a front portion 516, a back portion 518, and a crotch portion 526 extending between and connecting the front portion 516 and the back portion 518. Training pant 520 includes a bodyside liner 530 (facing away from the view depicted in FIG. 2) and an absorbent core 535 located between the bodyside liner and the outer cover 532. The training pant 520 has a pair of opposite longitudinal side edges 528 and 528′ that extend between a back waist edge 538 and a front waist edge 540. Training pant 520 also includes a pair of longitudinally-extending leg cuffs 536 and 536′. Leg cuffs 536 and 536′ may be adapted to fit about the legs of a wearer in use and serve as a mechanical barrier to the lateral flow of body exudates.

The training pant 520 includes a pair of side panels 510. The side panels 510 comprise front side panels 534 and 534′ and back side panels 534a and 534a′. The pairs of front side and back side panels 534, 534′, 534a, 534a′ may be formed integrally with the back and front portions or alternately may be formed separately and subsequently attached. Front side and back side panels 534, 534′, 534a, 534a′ comprise a distal edge 568 extending along a length of each respective panel, that can be seen in the prior art pant to form an overlapping seam when fastened. Front side and back side panels have a first major (inner) surface 545, which contacts the wearer and an opposing second major surface 547 that faces outward, away from the wearer.

In typical prior art training pants, front side and back side panels are releasably attached to one another by means of a prior art fastening system 544 and 544′. The prior art fastening system is used to secure the training pant 520 around the waist of a wearer. The fastening system may comprise a pressure sensitive adhesive, or a hook-type material with corresponding loop material for either fastening system 544 or 544′, for example. The front portion 516 would include an outer cover 532 suitably selected to form a receptive fastening component to the fastening system 544. For example, if fastening system 544 comprises a hook-type material, the training pant front portion 516 would comprise a suitable nonwoven material selected to suitably engage with and couple to the selected hook-type material.

To increase comfort and wearability, front side and back side panels 534, 534′, 534a, 534a′ may be formed of an elastic material that stretches in a direction parallel to the lateral direction 14 of the training pant 520.

The overlapping nature of the closure system used in training pant 520 results in a prominent overlap region where the fastening system overlaps with its counterpart. It also creates a flap, along for example distal edge 568, which makes the training pant feel and look a bit like diapers. Ideally, a more streamlined pants side seam closure system would be more discreet, and would not have overlap of the diaper components, so as to present as thin, without noticeable flaps of distal edges.

### Improved Pant Side Seam Closure

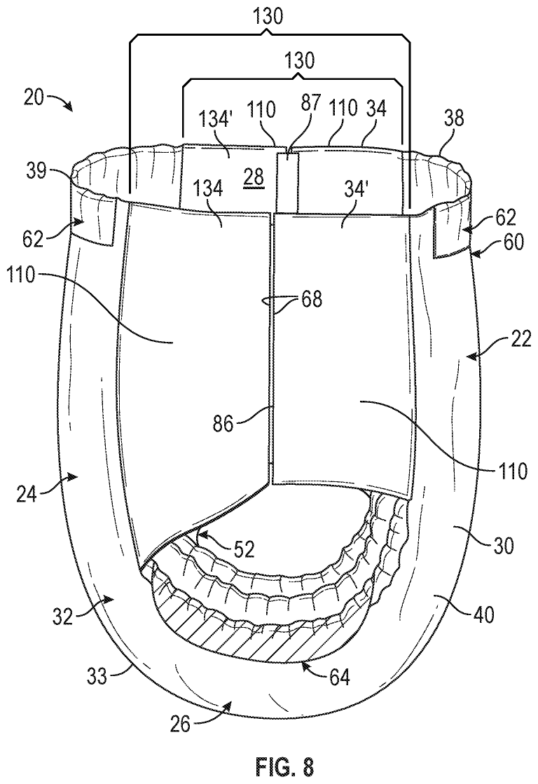

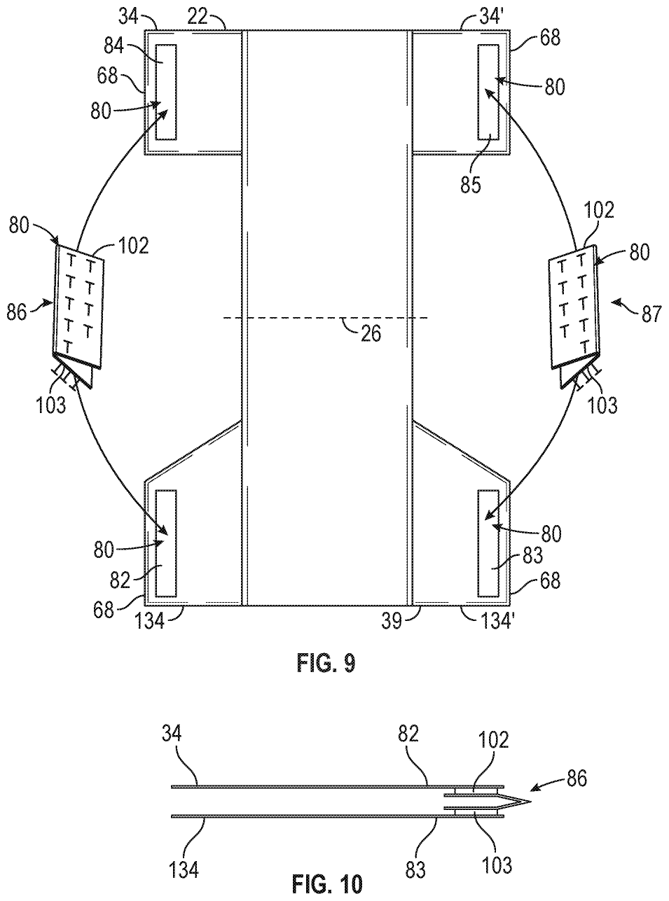

In reference to FIGS. 8 and 9, a training pant 20 according to embodiments of the present invention includes an improved lateral fastening system 80 for securing the training pant about the waist of the wearer, one on each lateral side of the wearer. In some embodiments, the improved fastening system may be thinner than pant-type lateral fastening systems of the prior art, and may resemble traditional underwear more than diapers in look and feel.

The improved fastening system comprises a bridging component 86 or 87 that is adapted to refastenably connect to fastening components 82, 83, 84, 85 provided on the front side and back side panels 34, 34′ 134, and 134′ respectively. The illustrated fastening system 80 includes first and second back side panel fastening components 82 and 83 and first and second front side panel fastening components 84 and 85 that are adapted to refastenably connect to the first and second bridging components 86 and 87. In some embodiments one or all of the fastening components 82, 83, 84, and 85 may be distinct from the front side and back side panels and attached by means of adhesive and/or thermal or ultrasonic bonding as is well known in the art. In other embodiments, the front side and back side panels themselves act as fastening components, as they may be made out of a material, such as a loop-type material, amenable to coupling with a hook-type fastening system on the bridging component. In such embodiments, the training pant 20 would include an outer cover 32 suitably selected to form a receptive fastening component to the bridging components 86 and 87. For example if bridging components 86 and 87 comprises a hook-type material, the side panels 34, 34′, 134, and 134′ might comprise a nonwoven material selected to suitably engage with and couple to the selected hook-type material.

In some embodiments, one surface of each of the first and second back side panel include discreet fastening components 82 and 83 (FIG. 9) and first and second front side panel fastening components 84 and 85. These fastening components comprise a plurality of engaging elements that are adapted to releasably engage and disengage the engaging elements of the bridging components 86 and 87.

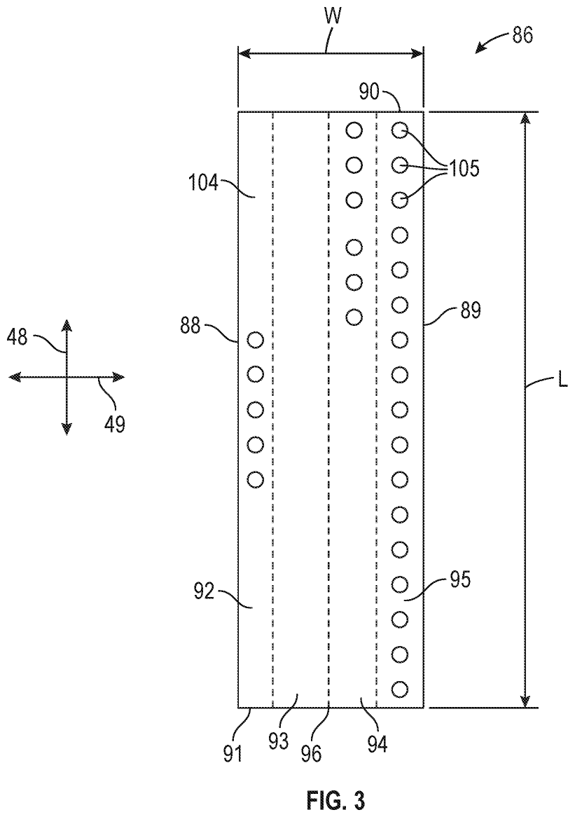

An embodiment of a bridging component 86 is depicted in FIG. 3. Bridging component 87, which would be used on the opposite lateral side of a pant, is in most embodiments of identical construction, so only bridging component 86 will be described herein. Bridging component 86 is generally rectangular, having a length € and a width, w, but may comprise any variety of shapes and features, including elliptical, clipped corners, and the like. Bridging components on the same pant may have the same, substantially same, or different dimensions and shape. Bridging component 86 comprises a pair of longitudinally opposed first and second edges, 88 and 89 and a pair of transversely opposed third and fourth edges 90 and 91. For ease of description of various aspects of the invention, bridging component 86 can be divided into four hypothetical quadrant lanes (92-95) that extend across the width between the opposed first and second edges 88 and 89 with a centerline 96 being defined at the boundary between the second and third quadrant lane 93 and 94, respectively.

Bridging component 86 includes fastening members 105 for releasably fastening the first and second front side panels 34 and 34′ to first and second back side panels 134, 134′, respectively. In some embodiments, fastening members 105 may comprise hook-type fasteners. In such case, the surface of the side panels on the pant (side panels side panels 34, 34′, 134, and 134′) would be provided with suitable complementary fastening components, e.g., loop material. In another embodiment, bridging component 86 may comprise loop type fasteners and the mating fastening components 82-85 (in reference to FIG. 9) each comprise complementary hook type fasteners. Loop type fasteners typically comprise a fabric or material having a base or backing structure and a plurality of loop members extending upwardly from at least one surface of the backing structure. The loop material can be formed of any suitable material, such as acrylic, nylon or polyester, and can be formed by methods such as warp knitting, stitch bonding or needle punching.

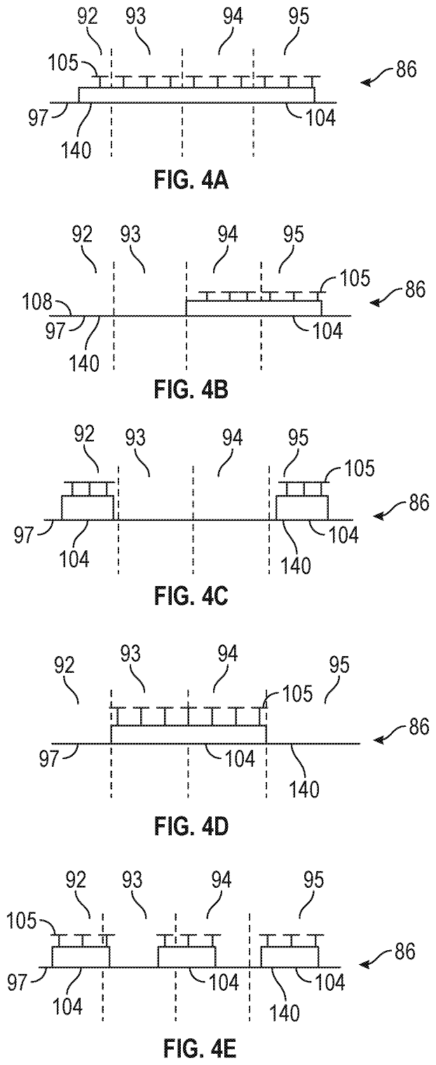

FIG. 4A-E shows various profile views various embodiments of bridging component 86. Bridging component 86 may comprise a bridging component backing 97 to which the material carrying the fastening members may attached. Since a body side surface 140 of bridging component backing 97 contacts the skin of the wearer when in use, it may be comprised of a soft material such as non-woven, such materials are well known in the art. To increase comfort, bridging component backing 97 may be formed of a breathable material. Bridging component backing 97 may be provided as a laminate and/or may optionally be elastic. Backing 97 may be the same or substantially the same size of as backing 104 that is carrying the fastening members 105. Alternately, bridging component backing 97 may be larger than the backing 104 carrying the fastening members 105, extending beyond the edges such that no portion of backing 104 is in contact with a wearer of a pant.

Backing 104 and the hook projections 105 may be of a construction known in the art. Hook type fasteners typically comprise a fabric or material having a base or backing structure and a plurality of hook members extending upwardly from at least one surface of the backing structure. Suitable hook material can be molded or extruded of nylon, polypropylene or another suitable material. Suitable single-sided hook materials for the bridging component 86 or the mating fastening components 82-85 are available from 3M Company of St. Paul, Minn. under product name CHK-05810. In some embodiments, hook backing material is sufficiently soft to eliminate need for bridging component backing.

To increase comfort, hook backing 97 may be breathable. Breathable hook backing coupled with breathable bridging component backing 104 would confer excellent overall breathability to the bridging components, improving air circulation and heat transfer. Examples of breathable hook tape include reticulated type fasteners such as those described by Chandrasekaran et al. in U.S. Pat. No. 9,649,824, the entire contents of which are incorporated herein. Other breathable hook tapes may be employed as is known to those of skill in the art.

To improve aesthetics and/or comfort, bridging components can have a minimal thickness, thereby reducing bulk, which could be uncomfortable to a wearer and/or could diminish the underwear look of the pant. In some embodiments the thickness of bridging component 86 ranges from about 30μ to about 900μ.

FIGS. 4B through 4E depict various embodiments alternative constructions of bridging component 86, each comprising hook type fastening members 105. Hook type members 105 are shown projecting outward from backing 104. Backing 104 is attached to a soft bridging component backing 97 (already described) with adhesive and/or ultrasonic or thermal bonding as is known in the art. Hook type members 105 may be provided in a variety of patterns across the backing 97. In the embodiment shown in FIG. 4A, hook type members 105 are distributed over substantially all the surface area of the bridging component backing 97 and extend across all four hypothetical quadrant lanes (92-95, depicted as dashed lines). In an alternate embodiment, depicted in FIG. 4E, hook type members 105 are provided in rows across all four hypothetical quadrant lanes. In alternate embodiments, hook type members 105 may be distributed over less than substantially all surface area of the bridging component backing 97. For example, as depicted in FIG. 4B, fastening members 105 may be provided on one side of the bridging component 86 and 87 thereby creating a flange 108 that may be used to attach the bridging component to any of the first or second front or back side panels 34, 34′, 134, 134′ with adhesive and/or ultrasonic or thermal bonding as is well known in the art. For example, fastening members, shown as hook type members 105, may be provided in quadrant lanes three and four only, alternately in quadrant lane three exclusively and further alternatively in quadrant lane four exclusively. In some embodiments, keeping quadrant free of hook creates an effective flange 108 that may be attached to the pant as described above (ultrasonic, adhesive, etc.). Alternatively, a flange is not provided and the bridging component attachment to the side panels occurs in regions comprising fastening members. In these embodiments, attachment may be achieved through the mating of the complementary fastening components and bridging component fastening members alone or a combination with adhesive and/or thermal or ultrasonic bonding as is well known in the art.

In other alternative embodiments, hook type members 105 may be provided at exterior edges of bridging component 86 such as in quadrant lanes one and four and two and three as depicted in FIGS. 4C and 4D, respectively. The embodiment shown in in FIG. 4C may be beneficial in the event that the front side and back side panel do not abut (described below) adequately to cover hooks provided on the bridging component 86 thus exposing potentially irritating textures to the wearer or the wearer's caregiver.

Soft bridging component backing 97 can be seen extending slightly beyond the perimeter of the backing 104 carrying the fastening members 105 (FIGS. 4A-4E). In some embodiments bridging component backing 97 is substantially the same size as the backing 104 carrying the fastening members 105. In other embodiments bridging component backing 97 is larger than the backing 104 carrying the fastener members 105.

The overall dimension of the bridging components 86 is not strictly limited. However, for ease of use and aesthetics, bridging component 86 is desirably sized to be concealed behind the front side and back side panels 34, 34′, 134, and 134′ when in use. As shown in FIG. 8, bridging components 86 and 87 may have a length l that is less than the length of the distal edges 68 of the front side and back side panels 34, 34′, 134, and 134′ so as to be concealed when fastened to a wearer. As will be appreciated, bridging components may have any length l that is less than the distal edges 68. Alternately, the length l may be greater than the distal edges 68. Bridging components 86 and 87 should be of a size adequate to remain secured to the wearer during normal physical activity.

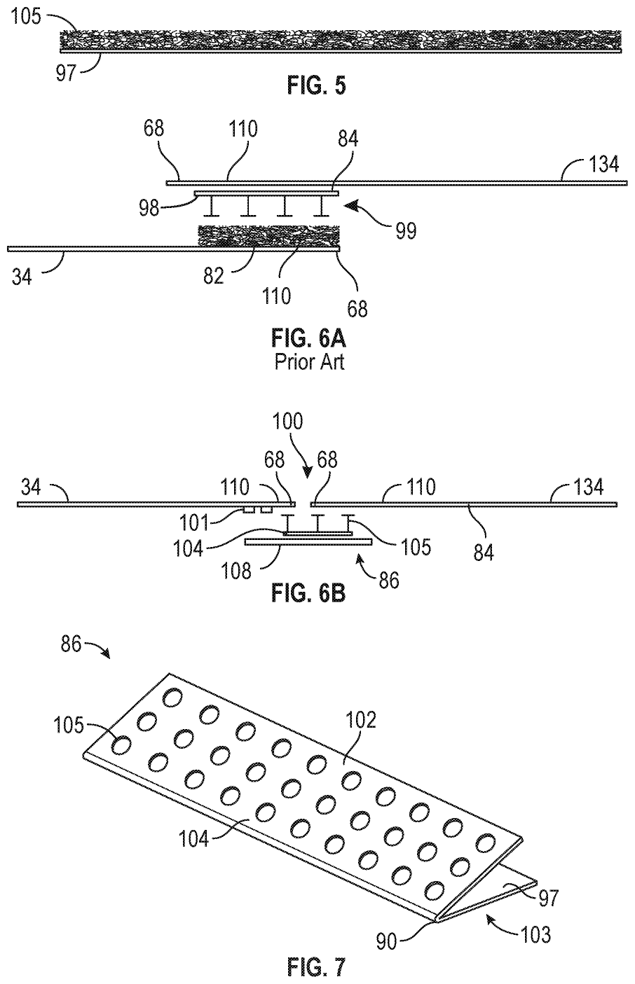

FIG. 5 illustrates an alternate embodiment of the bridging component 86 wherein the fastening members 105 comprises a loop type fastener. Such fasteners are known in the art. In these embodiments, front side and back side panels 34, 34′, 134, and 134′ may be provided with complementary mating fastening components, such as, e.g., hooks, as fastening components 82-84 (in reference to FIG. 9). Such fastening mechanisms are described in U.S. Pat. No. 5,888,607 to Seth et. al issued Mar. 3, 1998, the entire contents of which are herein incorporated by reference.

Seams formed utilizing embodiments of the bridging components described herein desirably eliminate the overlapping flaps that the fastening systems of prior art training pants typically include. A prior art fastening system 99 is illustrated in FIG. 6A where front side and back side panels 34, and 134 are shown coupled together via a releasable connection formed between a fastening component 84 provided on the back side panel 134 and a fastening component 82 provided on the front side panel 34. The nature of the prior art fastening systems creates an overlap 98 with a portion of the back side panel 134 undesirably overlapping a portion of the front (also shown in FIG. 1) thereby imparting a diaper-like look to the training pant.

In contrast, as depicted in FIG. 6B, embodiments of the bridging components of the present invention can be employed to produce low profile seams and aesthetically pleasing butt-joints 100, with the distal edges 68 of front side and back side panels 34, 34′, 134, and 134′ (only one side shown) abutted up next to each other 100 so that very little or no overlap is formed between the front side and back side panels 34, 34′, 134, and 134′. In this embodiments' configuration, the outer surface 110 of front side and back side panels 34, 34′, 134, and 134′ remain in the same plane even when engaged with the bridging component 86. In the embodiment shown, bridging component 86 includes a flange 108, a portion of which is adhered to a portion of the front side panel 34 near the distal edge 68 by adhesive and/or thermal or ultrasonic bonding 101. A portion of the bridging component 86 bearing fastening members 105 extends past the distal edge 68 of the front side panel 34 for engagement with a fastening component 84 provided on the back side panel 134. In the embodiment shown, back side panel 134 itself functions as fastening component 84 and is configured to releasably engage the hook type members 105 of the bridging component 86, for example, with loops (not shown). It will be appreciated that FIG. 6B depicts one side of a training pant, a second bridging component and second fastening component would be provided on the opposite side to enclose the training pant around the waist of the wearer.

FIG. 8 depicts a training pant 20 with the first and second front side and back side panels 34, 34′, 134, and 134′ fastened utilizing an embodiment of the bridging component 86 (and 87). First and second bridging components 86 and 87 are seen fastened on the inner surface (body side) 28 of the training pant 20. The majority of the surface area of the bridging components 86 and 87 is concealed (as viewed from outside the pant, if worn by a wearer) by the front side and back side panels 34, 34′, 134, and 134′. The distal edges 68 of the front and back side panels 34, 34′, 134, and 134′ are seen to be held in close proximity to each other forming a butt joint. In the embodiment shown, the distal edges 68 do not overlap. However, it should be appreciated that a small amount of overlap may occur without diminishing the low profile seam appearance achieved with use of the bridging components described herein. Even with a small overlap, training pant 20 still resembles underwear, unlike the prior art flap described previously.

Training pant 20 can be applied to a child in diaper or pant fashion. To apply in diaper fashion, the side panels are unfastened from the bridging components, which allows the training pant to be opened and laid flat out and the training pant applied to the child in a manner similar to a diaper. To apply in a training pant fashion, i.e., with side panels fastened to bridging components, the child's legs are placed through the leg openings 52 and the entire pant pulled up around the waist. It will be appreciated that once the training pant 20 is applied to the child, it can be pulled down and up without need for unfastening the front and back side panels 34, 34′, 134, and 134′. In this regard, the training pant 20 functions like underwear. With the low-profile butt joint achieved with use of the inventive fastening system, the pant also has the appearance of underwear.

First and second bridging components 86 and 87 may have sufficient pliability in the transverse and/or longitudinal axes to allow folding and/or bending (shown in FIG. 7) to facilitate automated production of pre-sealed training pants. In FIG. 7, bridging component 86 is folded along the longitudinal axis at approximately the centerline 96 (in reference to FIG. 3) such that the fastening members 105 face outward and the bridging component backing 97 faces inward. This creates a first fastening surface 102 and a second fastening surface 103 that are positioned to make fastening connection with the first or second front side or back side panels 34, 34′, 134, 134′. It will be appreciated that the way the bridging component is folded is not limiting. The only requirement is that the first and second fastening surfaces 102 and 103 are available to make fastening contact with the front side and back side panels 34, 34′ 134, 134′. Alternatively, bridging component can be provided pre-fastened to only one of the front side and back side panels 34, 34′ 134, 134′ or unfastened to any surface of the absorbent articles and instead provided as a discrete component. In these embodiments, the user would seal the training pant 20 prior to placing on a child or could apply the training pant 20 in a fashion like that of a traditional diaper.

The folding of bridging component 86 can advantageously be employed in a manufacturing process to provide pre-fastened training pants 20. The manufacture of diapers and training pants is well known in the art. Briefly, and in reference to FIG. 8, training pants are typically start as a continuous web connecting the front waist edge 40 of one pant to the back-waist edge 38 of the adjacent pant. The crotch portion of individual pants are cut, then folded at centerline (shown as dashed line in FIG. 9) in the crotch portion 26. Bridging components can be provided as a continuous roll, folded, and subsequently cut into the desired dimension at the time of manufacture of the pant.

Individual bridging components can be introduced while the training pants are in the unfolded state as shown in FIG. 9. Bridging components 86 and 87 should be oriented so that at least one of the first or second fastening surfaces 102 or 103 (in reference to FIG. 7) makes fastening contact with its corresponding mating surface of fastening component 82, 83, 84, or 85 on the first or second front side or back side panel 34, 34′, 134, 134′. In one embodiment, webbing and/or individual training pant 20 and bridging components 86 and 87 are brought into proximity of each other such the first fastening surface 102 of each of the bridging components 86 and 87 align with the mating surfaces of fastening components 84 and 85 of the front side panels 34 and 34′. In the embodiment shown, first surface 102 includes hook type fastening members 105. In this embodiment, first fastening surface 102 may be releasably attached via hook and loop type fastening. Alternately, first surface 102 may be secured by additional means such as adhesive and/or thermal or ultrasonic bonding. In other embodiments, first surface is provided as a flange 108 (shown in FIG. 4B). In these embodiments, flange 108 may be provided without fastening members and be non-releasably attached to mating surfaces by means of adhesive alone or in combination with thermal and/or ultrasonic bonding.

With first surfaces 102 thus attached to first and second front side panels 34 and 34′, second surfaces 103 of first and second bridging components 86 and 87 are exposed and available for releasable fastening engagement with fastening components 82 and 83 of back side panels 134 and 134′. As shown in FIG. 10, when training pant 20 is folded at centerline (shown as dashed line in FIG. 9) in crotch portion 26, fastening components 82 and 83 come into contact with second surfaces 103 of bridging components 86 and 87 and engage to form a pant with two non-overlapping side seams at both sides. At this point, fastened training pants 20 may be packaged.

Thus, embodiments of the Absorbent Article With Refastenable Fastening System are disclosed. One skilled in the art will appreciate that the present invention can be practiced with embodiments other than those disclosed. The disclosed embodiments are presented for purposes of illustration and not limitation, and the present invention is limited only by the claims that follow.

1. An absorbent article, comprising:
an absorbent chassis having a front portion, back portion, and crotch portion the crotch portion extending between and connecting the front portion and back portion, the absorbent chassis having first and second major longitudinal edges;
first and second laterally opposed side panels, connecting the front portion to the back portion along a length of the first and second major longitudinal edges, to define first and second leg openings in the absorbent article;
wherein the first and second side panels comprise:
a front side panel having a distal edge;
a rear side panel having a distal edge; and,
a bridging component releasably coupling the front and rear side panels in a non-overlapping configuration.

2. The absorbent article of claim 1, wherein the first and second front side panels and first and second back side panels comprise at least one fastening component for refastenably engaging a bridging component.

3. The absorbent article of claim 1, wherein the fastening component comprises a loop-faced material.

4. The absorbent article of claim 3, wherein the bridging component comprises a backing with hook members extending therefrom.

5. The absorbent article of claim 1, wherein the bridging component comprises a hook-faced tape.

6. The absorbent article of claim 1, wherein the bridging component comprises a pressure sensitive adhesive-backed tape.

7. The absorbent article of claim 1, wherein either the front side panels or back side panels comprise a pressure sensitive adhesive for refastenably engaging the bridging component associated with the first or side panels.

8. The absorbent article of claim 4, wherein the thickness of the bridging components ranges from about 40 microns to about 900 microns.

9. The absorbent article of claim 1, wherein the bridging component further comprises a bridging component backing that contacts the skin of a wearer of the absorbent article that is sufficiently soft to provide a non-irritating interface for a wearer's skin.

10. The absorbent article of claim 9, wherein the bridging component backing is breathable.

11. The absorbent article of claim 1, wherein non-overlapping configuration comprises first and second butt-joints formed by the distal edges of the front and rear side panels of the first and second side panels, with the bridging component straddling the butt-seam.

12. The absorbent article of claim 1, wherein the bridging component comprises fastening members extending from a backing.

13. The absorbent article claim Error! Reference source not found. 2, wherein a body side surface of the backing is sufficiently soft to avoid irritating the skin of a wearer.

14. The absorbent article of claim 12, wherein the backing comprises polyolefin polymers.

15. The absorbent article of claim 1, wherein the bridging component is breathable.

16. The absorbent article of claim 1, wherein the bridging components comprise a laminate, the laminate comprising a hook tape having a backing with hooks extending therefrom, wherein the laminate is breathable.

17. The absorbent article of claim 16, wherein a body side surface of the bridging component backing is sufficiently soft to avoid irritation of the skin of a wearer.

18. The absorbent article of claim 1, wherein at least a portion of the bridging component is bonded to the first or second major longitudinal edge of the absorbent chassis with adhesive and/or thermal or ultrasonic bonding.

19. The absorbent article of claim 16, wherein the bridging components associated with the first and second side panels have a length and a width, and wherein the length is greater than the width, and wherein the hook-side of the bridging component comprises at least one quadrant lane that does not include hooks.

20. The absorbent article of claim 19, wherein the at least one quadrant lane that does not include hook defines a flange, and wherein the flange is coupled to either the front or rear side panel with adhesive and/or thermal or ultrasonic bonding.

21. The absorbent article of claim 1, wherein the bridging component and front side panel and rear side panel are configured in a nonoverlapping butt joint.

22. A method of making an absorbent article, the method comprising:
coupling first and second side panels to an absorbent chassis, the absorbent chassis having a front portion, back portion, and crotch portion, the crotch portion extending between and connecting the front portion and back portion, and having first and second major longitudinal edges, and wherein the first and second side panels are coupled to absorbent chassis along lengths of the first and second major longitudinal edges, to define first and second leg openings in the absorbent article;
wherein the first and second side panels comprise:
a front side panel having a distal edge;
a rear side panel having a distal edge; and,
a bridging component coupling the front and rear side panels in a non-overlapping configuration.

23. The method of claim 22, wherein the bridging components associated with the first and second side comprise a fold along a longitudinal axis extending between the distal edges of the respective front side and rear side panels.

24. The method of claim 22, wherein the front and rear side panels associated with the first and second side panels comprise a backing having loop fastening component on one major surface thereof, and the bridging component associated with the first and second side panels comprise hook material.

25. The method of claim 22 wherein coupling comprises joining with adhesive and/or thermal or ultrasonic bonding.

26. The method of claim 22, wherein the front and rear side panels of the first and second side panels are coupled with the bridging component in butt-seam configuration, with the bridging component straddling the respective front and rear side panels.

27. The method of claim 22, wherein fewer than four quadrant lanes of the first and second bridging components comprises fastening members.

28. The method of claim 22, wherein the fastening members comprise hook-type fasteners.

29. The method of claim 16, wherein the hook-type fasteners are provided on approximately the third and fourth quadrant lanes, at least a portion of the first and second quadrant lanes constituting a flange.

30. The method of claim 22, wherein the fastening member comprises loops.

31. The method of claim 29, wherein the flange is attached to one of the first or second front or first second back panels.

32. The method of claim 31, wherein the flange is attached by adhesive and/or thermal or ultrasonic bonding.

33. The method of claim 22 wherein, the bridging components are folded on a transverse axis thereof prior to being applied to the first and second back panels.

34. A side panel for a pull-up type absorbent article, comprising:
a first panel having first and second major surfaces and distal edge;
a second panel having first and second major surfaces and a distal edge;
a bridging component coupled to the first major surface of the first panel and the first major surface of the second panel;
wherein the bridging component holds the distal edges of the first and second panels together in a butt-joint configuration.

35. The side panel of claim 34, wherein the bridging component comprises a hook tape, and wherein the first major surfaces of the first and second panels comprise loop material.

36. The side panel of claim 35, wherein the butt-joint configuration comprises a resealable seam.

37. The side panel of claim 34, wherein the bridging component comprises a length of pressure-sensitive adhesive-backed tape.

38. The side panel of claim 37, wherein the bridging component straddles portions of the first major surfaces of the first and second panels.

39. The side panel of claim 34, wherein first and second panels are coupled to the bridging component and no portion of the first or second major surface of the first or second panels contacts the first or second major surface of the other one.

40. The side panel of claim 34, wherein the first and second panels such that the bridging component straddles areas of the first major surface of the first and second panels, and the first and second panels do not overlap.
